(19) United States
(12) Patent Application Publication
(10) Pub. No.: US 2022/0133945 A1
(43) Pub. Date: May 5, 2022

(54) Acrylic Polymer and Adhesive Compositions

(71) Applicant: Henkel IP & Holding GmbH, Dusseldorf (DE)

(72) Inventors: Michael Krejsa, Rocky Hill, CT (US); Mary Palliardi, Rocky Hill, CT (US); Andrew Messana, Rocky Hill, CT (US)

(21) Appl. No.: 17/570,512

(22) Filed: Jan. 7, 2022

Related U.S. Application Data
(63) Continuation of application No. PCT/US2020/070027, filed on May 6, 2020.
(60) Provisional application No. 62/872,868, filed on Jul. 11, 2019.

(51) Int. Cl. A61L 15/58; C08F 220/18; C09J 7/38; C09J 7/25; A61L 15/24

(52) U.S. Cl. CPC A61L 15/58; C08F 220/1808; C09J 7/385; C09J 2467/006; A61L 15/24; C09J 2433/00; C09J 7/255

(57) ABSTRACT

Acrylic polymers and pressure sensitive adhesive compositions prepared from (i) from about 1 to about 10 wt % of a carboxy functional monomer component, (ii) from about 50 to about 90 wt % of a low Tg alkyl acrylate monomer component, (iii) from about 1 to about 20 wt % of a tertiary amine functionalized alkyl acrylate monomer component, and (iv) optionally, up to 50 wt % of a vinyl ester monomer component are disclosed. The acrylic polymers and pressure sensitive adhesives are ideally suited for skin contact applications, particularly as diagnostic patches, transdermal drug-delivery patches, and dermal patches for delivery of skin actives.

### FIELD OF THE INVENTION

The invention relates to acrylic polymers and pressure sensitive adhesive compositions, and end use applications thereof, which are ideally suited for skin contact applications including, but not limited to, adhesives for adhering medical devices to skin, diagnostic patches, transdermal drug-delivery patches, and dermal patches for delivery of skin actives.

### BACKGROUND OF THE INVENTION

Adhesive compositions find widespread use in medical segment field, e.g., various tapes, bandages and drug delivery devices. These devices have an adhesive layer formed on at least one surface of a substrate and are adhered to the target skin surface via the adhesive layer. The adhesive is a pressure sensitive adhesive, which is permanently tacky at room temperature, holds the adhered article to the skin with gentle pressure, and is easily removed without causing pain or leaving adhesive residue. The devices, depending on the purpose of the application, may be required to secure adhesion to the skin surface for an extended time period. These devices are typically in the form of ostomy bags, bandages, diagnostic vehicles, and transdermal patches.

While pressure sensitive adhesives for application to the skin are known, there is an ongoing demand and continuing need in medical applications. The current invention addresses this need in the art.

### BRIEF SUMMARY OF THE INVENTION

The present invention relates to acrylic polymers and adhesive compositions, and articles of manufacture comprising the acrylic polymers and adhesive compositions, particularly in medical skin contact devices.

One aspect of the invention is directed to an acrylic polymer prepared from (i) from about 1 to about 10 wt % of a carboxy functional monomer component, (ii) from about 50 to about 90 wt % of a low Tg alkyl acrylate monomer component, (iii) from about 1 to about 20 wt % of a tertiary amine functionalized alkyl acrylate monomer component, and (iv) optionally, up to 50 wt % of a vinyl ester monomer component.

Another aspect of the invention is directed to a pressure sensitive adhesive composition comprising an acrylic polymer prepared from (i) about 1 to about 10 of a carboxy and/or hydroxy functional monomer; (ii) from about 50 to about 90 wt % of a low Tg alkyl acrylate monomer component, (iii) from about 1 to about 20 wt % of a tertiary amine functionalized alkyl acrylate monomer.

Still another aspect of the invention is directed to a medical skin contact device comprising an adhesive layer containing the pressure sensitive adhesive composition of above, and a backing layer. The pressure sensitive adhesive composition in the medical skin contact device provides long term adhesion and shear performance.

Yet another aspect of the invention is directed to a method of forming an acrylic polymer comprising the steps of (a) forming a monomer mixture of 1 to about 10 of (i) a carboxy and/or hydroxy functional monomer; (ii) from about 50 to about 90 of a low Tg alkyl acrylate monomer component, (iii) from about 1 to about 20 wt % of a tertiary amine functionalized alkyl acrylate monomer component in a vessel; (b) adding a solvent and a crosslinker to the vessel; (c) heating the vessel; and (d) isolating the acrylic polymer.

### DETAILED DESCRIPTION OF THE INVENTION

As used herein, the term “pressure-sensitive adhesive” or “PSA” refers to a viscoelastic material which adheres instantaneously to most substrates with application of slight pressure and remains permanently tacky. A polymer is a pressure sensitive adhesive within the meaning of the term as used herein if it has the properties of a pressure sensitive adhesive per se or functions as a pressure sensitive adhesive by admixture with other components. The pressure sensitive adhesive of the invention may be used in any number of applications, e.g., labels, tapes, medical skin contact device tapes for diagnostics and transdermal purposes.

The term “transdermal” refers to application on or to the skin whereby the skin is used for diagnostic procedures, such as the monitoring of blood chemistry, or as a portal for the administration of drugs by topical application.

The terms “skin,” “derma.” and “epidermis” are used interchangeably unless specifically stated otherwise.

The term “patient” is used herein to include animals, both human and non-human, including companion animals such as dogs, cats and horses and livestock such as cattle and swine. Agricultural and horticultural applications are also contemplated.

Weight percent, “wt %,” indicates the weight percent based on the total weight of the components in the polymer.

The pressure sensitive adhesive is formed from an acrylic polymer. The pressure sensitive adhesive may be neat acrylic polymer or may be combined with tackifiers, plasticizers or other additives to provide the properties of pressure sensitivity.

The acrylic polymer prepared from (i) from about 1 to about 10 wt % of a carboxy functional monomer component, (ii) from about 50 to about 90 wt % of a low Tg alkyl acrylate monomer component, (iii) from about 1 to about 20 wt % of a tertiary amine functionalized alkyl acrylate monomer component, and (iv) optionally, up to 50 wt % of a vinyl ester monomer component.

The (i) carboxy functional monomer component contain from about 3 to about 12 carbon atoms and include, among others, acrylic acid, methacrylic acid, itaconic acid, β-carboxyethyl acrylate, and mixtures thereof.

The (ii) low Tg alkyl acrylate monomer are those having a homopolymer Tg of less than about −30° C. Preferred alkyl acrylates for the acrylic polymer have up to about 18 carbon atoms in the alkyl group, preferably from about 4 to about 12 carbon atoms in the alkyl group. Alkyl acrylates for preparation of the acrylic polymer include methyl acrylate, butyl acrylate, amyl acrylate, hexyl acrylate, 2-ethylhexyl acrylate, octyl acrylate, isooctyl acrylate, decyl acrylate, dodecyl acrylates, isomers thereof, and combinations thereof. Particularly preferred are butyl acrylate, 2-ethylhexyl acrylate and/or isooctyl acrylate, most preferably 2-ethylhexyl acrylate.

The (iii) a tertiary amine functionalized alkyl acrylate monomer component is selected from 2-dimethyl aminoethyl methacrylate, 2-N-morpholinoethyl acrylate, 2-N-morpholinoethyl methacrylate, 2-diisopropylaminoethyl methacrylate, N-[3-(N,N-dimethylamino)propyl] methacrylamide, N-[2-(N,N-dimethylamino)ethyl]methacrylamide, N-[3-(N,N-dimethylamino)propyl] acrylamide, min. 95%, 2-(N,N-dimethylamino)ethyl acrylate, 2-(N,N-diethylamino)ethyl methacrylate, 2-(N,N-dimethylamino)ethyl methacrylate, 2-acryloxyethyltrimethylammonium chloride, and mixtures thereof.

The acrylic polymer may optionally comprise (iv) one or more vinyl ester monomers. Preferred are carboxy and/or hydroxy functional monomers. Useful vinyl esters include vinyl acetate, vinyl benzoate, vinyl-tert-butylbenzoate, vinyl chloroformate, vinyl cinnamate, vinyl decanoate, vinyl neodecanoate, vinyl pivalate, vinyl propionate, vinyl stearate, vinyl trifloroacetate, vinyl valerate, and mixture thereof, and particularly preferred is vinyl acetate.

The acrylic polymer may also comprise a hydroxy functional monomer component and derivatives thereof. The hydroxy functional monomer component is selected from the group consisting of hydroxyethyl acrylate, hydroxypropyl acrylate, hydroxyethyl methacrylate and hydroxypropyl methacrylate, and mixtures thereof. Alternatively, the acrylic polymer further comprises an anhydride functional monomer component and derivatives thereof. The anhydride functional monomer component is selected from the group consisting of maleic anhydride, nadic anhydride, methyl maleic anhydride, nadic methyl anhydride or like compounds. The addition of a hydroxy functional monomer or anhydride functional monomer component in the acrylic polymer provides improved shear performance of the adhesive. It is believed that an increase of ionic character of the hydroxy functional monomer or the anhydride functional monomer in the adhesive provides this improved shear performance.

In one preferred embodiment, the acrylic polymer is prepared (i) from about 1 to about 10 wt % of an acrylic acid, (ii) from about 50 to about 90 wt % of a 2-ethylhexyl methacrylate, (iii) from about 1 to about 20 wt % of 2-dimethyl aminoethyl methacrylate and (iv) up to about 50 wt % of vinyl acetate.

While a particular polymerization method is described in the examples, the acrylic polymer may be prepared by conventional polymerization methods familiar to those of skill in the art. These methods include, without limitation solution polymerization, suspension polymerization, bulk polymerization and emulsion polymerization. It may also be advantageous to reduce the residual monomer content, or remove or reduce solvent levels and/or other volatiles, following polymerization using methods which are known and conventional in the art.

One method of forming the acrylic polymer comprises the steps of:
(a) forming a monomer mixture of 1 to about 10 of (i) a carboxy and/or hydroxy functional monomer; (ii) from about 50 to about 90 of a low Tg alkyl acrylate monomer component, (iii) from about 1 to about 20 wt % of a tertiary amine functionalized alkyl acrylate monomer component in a vessel;
(b) adding solvent and an initiator to the vessel;
(c) heating the vessel; and
(d) isolating the acrylic polymer.
   Various initiators and solvents may be used to aid the polymerization process.

Initiators useful for polymerization include, 2,2′-azodi-(2-methylbutyronitrile, 2,2′-azobis-isobutyronitrile, 1,1′-azobis(cyclohexane-1-carbonitrile) and 2,2′-azobis-(2,4-dimethylvaleronitrile), and as examples of peroxide-based polymerization initiators there may be mentioned lauryl peroxide, benzoyl peroxide and di(tert-butyl) peroxide.

Solvents useful for polymerization include organic solvents, for example, ethyl acetate, acetone, hexane, cyclohexane, heptane, toluene, ethanol, and isopropyl alcohol, or a combination thereof.

The acrylic polymer, in its neat form, is viscoelastic and adheres instantaneously to stainless steel and synthetic skin (Vitro-Skin®, Portland, Me.) substrates.

In another embodiment, a pressure sensitive adhesive composition is formed comprising an acrylic polymer prepared (i) from about 1 to about 10 of a carboxy and/or hydroxy functional monomer; (ii) from about 50 to about 90 of a low Tg alkyl acrylate monomer component, and (iii) from about 1 to about 20 wt % of a tertiary amine functionalized alkyl acrylate monomer.

The acrylic polymer may be combined with plasticizers, tackifier and other additives to improve adhesive characteristics of the pressure sensitive adhesive. Examples of plasticizers include fatty acid esters of monohydric alcohols, such as cetyl octanoate, hexyl laurate, isopropyl myristate, isopropyl palmitate, butyl stearate and myristyl lactate; dibasic acid esters such as dioctyl adipate, diethyl sebacate, dioctyl sebacate and dioctyl succinate; and fatty acid esters of polyhydric alcohols such as propyleneglycol dicaproate, glyceryl trioctanoate, glyceryl tri(octanoate/decanoate), medium chain fatty acid triglycerides and the like, among which fatty acid esters such as isopropyl myristate, isopropyl palmitate.

Suitable tackifying agents include: (1) aliphatic hydrocarbons; (2) mixed aliphatic and aromatic hydrocarbons; (3) aromatic hydrocarbons; (4) substituted aromatic hydrocarbons; (5) hydrogenated esters; (6) polyterpenes; and (7) wood resins or rosins and hydrogenated forms thereof. The tackifying agent employed is preferably compatible with the polymer. The pressure sensitive adhesive may further include a rheology modifier, diluents, emollients, anti-irritants, opacifiers, fillers, such as clay and silica, pigments, preservatives, antioxidants or additives.

The pressure sensitive adhesive is viscoelastic and adheres instantaneously to various substrates with slight pressure and remains permanently tacky. Shear strength improves with the inventive polymer, indicating the polymer's utility for long-term wearable devices. Peel strength of the polymer remains similar to or improves, further indicating the utility to remain on dermis.

The pressure sensitive adhesive is useful in the manufacture of industrial tapes, films, and labels such as masking tapes, surface protection films, bookmarks, notepapers, price marking labels, promotional graphics materials, and the like; and medical skin contact devices, such as wound care dressings, EKG electrodes, athletic tape, ostomy bag, analgesic and transdermal patch, and the like.

An active may be incorporated into the pressure sensitive adhesive. The pressure sensitive adhesive may be advantageously formulated for use in wound care, transdermal or dermal drug or cosmeceutical delivery applications. The term transdermal refers to the use of the skin as a portal for the administration of drugs by topical application. The topically applied drug passes into and/or through the skin. Thus “transdermal” is used broadly to refer to the topical administration of a drug which acts locally, i.e., at the surface or within the skin, such as, for example, a blemish patch used to treat acne, and to the topical application of a drug which acts systemically by diffusing through the skin and entering the blood stream. The term “drug” is to be construed herein in its broadest sense to mean any agent which is intended to produce some therapeutic benefit. The agent may or may not be pharmaceutically active but will be “bioactive” in the sense that it has an effect on the human body. The agent may be used to treat or alter a condition, which may or may not be a pathological, i.e., a disease state. “Active”, “drug”, “bioactive agent,” “preparation,” “medicament,” “therapeutic agent,” “physiological agent” and “pharmaceutical agent” are used interchangeably herein and include substances for use in the diagnosis, cure, mitigation, arrest, treatment or prevention of a condition or disease state or to affect the structure or function of the body. Skin-wellness agents that function to e.g., soften and moisturize, are included in this term. The term “treatment” is used broadly to encompass prevention, alteration, cure and control of the condition.

Examples of drugs include general anesthetics, hypnotics/analgesics, anti-epileptic agents, antipyretic analgesic anti-inflammatory agents, steroidal anti-inflammatory drugs, stimulants/analeptics, anti-motion sickness agents, psychoneurotic drugs, local anesthetics, skeletal muscle relaxants, autonomic nerve agents, antispasmodic drugs, anti-Parkinson drugs, antihistamines, cardiac stimulants, anti-arrhythmia drugs, diuretics, antihypertensive agents, vasoconstrictors, vasodilators, anti-arteriosclerotic agents, respiratory stimulants, antitussive expectorants, peptic ulcer treatment agents, cholagogues, hormone agents, urogenital and anal drugs, anti-asthmatic drugs, parasitic skin disease drugs, emollients, vitamins, inorganic preparations, hemostatic drugs, anti-coagulants, liver disease drugs, drug addiction agents, anti-gout agents, anti-diabetes agents, anti-malignant tumor agents, radioactive medicines, Chinese herbal preparations, antibiotics, chemotherapeutic agents, vermifuges/antiprotozoal agents, narcotics, and the like.

Examples of cosmetic ingredients include whitening ingredients such as ascorbyl palmitate, kojic acid, lucinol, tranexamic acid and oil-soluble licorice extract, wrinkle preventers such as retinol, retinoic acid, retinol acetate and retinol palmitate, circulation improving ingredients such as vitamin E, tocopherol acetate, capsaicin and vanillylamide nonylate, antimicrobial ingredients such as isopropylmethylphenol, light-sensitive elements and zinc oxide, and vitamins such as vitamin D2, vitamin D3 and vitamin K.

The content of a drug or cosmetic ingredient in the pressure sensitive adhesive for skin may be appropriately determined according to the type and purpose of use. Drugs and cosmetic ingredients may also be encapsulated together with absorption accelerators, or a retaining layer may be provided for the medical or cosmetic ingredients.

The pressure sensitive adhesive, alone or with an active, can be made in the form of a device such as a medical tape, a medical patch, a medical sheet, a medical dressing, a transdermal patch, industrial tape, industrial sheet or any other form known to those skilled in the art. Various shapes and sizes of the device are contemplated.

Still another aspect of the invention is directed to a device comprising the pressure sensitive adhesive layer and a backing layer. In one embodiment, the device further comprises a release liner. In a preferred embodiment a device is formed with a pressure sensitive adhesive layer, a distal backing layer and a proximal release liner.

The portions of the device that are not in contact with the skin are covered by a backing. The distal backing layer, in use, defines the side of the device that faces the environment, i.e., distal to the skin. The backing serves to protect the device, including additional active contents, within the device, from the environment by providing an impenetrable layer that prevents loss of the adhesive and the actives, if any, to the environment. Thus, the material chosen should be substantially impermeable to the actives and adhesives. Advantageously, the backing material can be opaque to protect the contents from degradation from exposure to light. As the backing substrate, both a non-porous film and a porous film may be used. It may be desirable that the backing have a relatively high vapor transmission rate, since this results in the reduction of moisture buildup on the skin beneath the device and in a corresponding reduction in the amount of skin maceration that occurs. Conversely, to enhance active flux, an occlusive backing may be selected. Further, the backing layer should be capable of binding to and supporting the other layers of the device yet should be pliable to accommodate the movements of a person using the device since a stiff backing may cause mechanical irritation. In order to maintain the health of the covered skin during long term wear (e.g., for periods in excess of a day), it is also desirable that the backing have a relatively high permeability to oxygen. As the backing is in contact with the adhesive and possible actives, it is important that the backing be stable to such components in order that the backing retains its structural integrity and conformability. It is also desirable for the backing to be heat sealable at a relatively low temperature to a variety of other polymeric substrates.

The backing constituting the adhesive for skin of the present invention is not subject to any particular limitation as long as it can support the adhesive layer. Backings that have found use in drug delivery devices, and which can be used in the practice of this invention include, with or without modification, metal foils, metalized polyfoils, composite foils or films containing poytetrafluoroethylene (TEFLON®)-type materials or equivalents thereof, polyether block amide copolymers, polyurethanes, polyvinylidene chloride, nylon, silicone elastomers, rubber-based poylisobutylene styrene, styrene-butadiene and styrene-isoprene copolymers, polyethylene, polyester, and other such materials used in the art of transdermal drug delivery. Particularly preferred are thermoplastic polymers such as polyolefins, for example polyethylene and polypropylene, and polyesters such as polyethyleneterephthalate. In order to increase adhesion with the pressure-sensitive adhesive layer, the backings may be subjected to surface treatment such as corona treatment or plasma discharge treatment, or to anchor coat treatment with an anchoring agent.

The above-mentioned backing has a thickness of 10-100 μm, preferably 20-40 μm, so that uncomfortableness is not felt at the site where the device is adhered to the skin.

It is also preferable to adjust the above-mentioned backing to have a tensile strength of 100-900 kg/cm² and 100% modulus of 10-100 kg/cm², to provide fine skin-followability of device for application to skin.

The proximal release liner or peelable film covers the skin-facing or proximal side of the device until the device is used. A silicone-coated film is typically used for such applications. Just prior to use of the device, the proximal release liner is removed to expose the pressure sensitive adhesive layer for contact and adhesion to the skin surface. Thus, the proximal release liner is adapted to be removed from the device and should strip off the adhesive surface with minimal force.

In one embodiment, the release liner of a first patch also serves as the backing layer of a second patch. This design allows patches to be manufactured in a stacked format and dispensed to the patient in this manner. The first patch is removed, and applied to the skin, with no excess waste generated for disposal.

The pressure sensitive adhesive may be applied from organic solution, aqueous dispersion, or from a melt onto the backing. Matrix patches are particularly preferred embodiments since they are easier to manufacture than liquid reservoir patches and are more comfortable and convenient to wear. A matrix patch device is a unit dosage form of an active in the pressure sensitive adhesive. Generally, the device will be in the form of a patch of a size suitable to deliver a preselected amount of active through the skin. A surface area in the range of 1 to 200 cm² is contemplated and preferred sizes are 5, 10, 15, 20, 25 and 30 cm². The thickness may vary over a wide range, typically from about 1 to about 5 mil, preferably 3 to 4 mil thick.

The device can be prepared by using conventional methods. For example, a matrix device can be manufactured by preparing a coating formulation by mixing a solution of the pressure sensitive adhesive in a solvent with an active to form a homogeneous solution or suspension; applying the adhesive to a substrate (a backing or a release liner) using well known knife or bar or extrusion die coating methods; drying the coated substrate to remove the solvent; and laminating the exposed surface to a release liner or backing. The matrix may further contain an active, depending on the intended use of the device.

The device of the invention is placed on the skin and allowed to remain for a time sufficient to achieve or maintain the intended purpose. The time that constitutes a sufficient time can be selected by those skilled in the art with consideration of the flux rate of the device of the invention and of the condition being treated, e.g., diagnostic procedure or deliver a therapeutic effect. Depending on the design of the patch and the condition to be treated, the patch will remain on the skin for up to an hour or more, up to about one week. In one embodiment, the patch is designed to remain on the skin at the application site for at least 24 hours, and up to 7 days. In another embodiment, the patch is designed to remain on the skin for 2 weeks or even up to a month.

The device, in general, must reliably adhere to the skin for at least 24 hours, and in some embodiments, greater than 2 days, 3 days, 7 days, 14 days, 21 days and even 30 days, after being attached to skin and must also adhere without peeling during perspiration and bathing. Also, for removal, they must be peelable with a degree of force that does not cause pain, and if the adhesion is stronger than necessary it can result in plucking of hairs and peeling of the corneum, as well as mechanical skin irritation by pulling of the skin. Consequently, this creates erythema that may persist for several days even after peeling, and therefore it is necessary to minimize this inconvenience. Moreover, it is important that no pressure-sensitive adhesive remain on the skin surface after the pressure sensitive adhesive sheet has been removed from the skin.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### EXAMPLES

### Example 1: Control Acrylic Polymer

An initial charge containing 434 g of 2-ethylhexyl acrylate and 13 g of acrylic acid was prepared and slowly charged to a flask containing 70 g of 2-ethylhexyl acrylate, 3 g of acrylic acid, 123 g vinyl acetate, 84 g Heptane and 90 g of ethyl acetate equipped with stainless steel stirrer, thermometer, condenser, water bath, and additional funnels. The initial charge was heated to reflux while stirring for about 3 hours with the simultaneous slow addition of AIBN. Heptane was added over to hours and refluxed an additional hour. Aluminum acetylacetonoate, 2,4-pentanedione, toluene, and isopropanol were added. At the end of this, the contents were cool and flushed with isopropyl alcohol and the crosslinked solids in solution were studied.

### Example 2: Acrylic Polymers

The acrylic polymers samples A, B, C, and D were made similarly as Example 1 Control, but with varying amounts of 2-dimethylethyl methacrylate (2-DMAEMA) and hydroxyethyl acrylate (HEMA), and maleic anhydride (MA). The weight percent of the monomers directly replace Control Sample's 2-ethylhexyl acrylate percentages.

TABLE 1
Control and Samples monomer replacements

| | 2-DMAEMA | HEMA | MA |
|---|---|---|---|
| Control 1 | 0 | 0 | 0 |
| Sample A | 2.5 | 0 | 0 |
| Sample B | 5.0 | 0 | 0 |
| Sample B1 | 5.0 | 2.5 | 0 |
| Sample C | 7.5 | 0 | 0 |
| Sample C1 | 7.5 | 2.5 | 0 |
| Sample D | 10 | 0 | 0 |
| Sample D1 | 0 | 0 | 10 |

### Example 3: Shear and Peel Adhesion

Shear adhesion was measured according PSTC No. 107 using a 1,000 gram (g) mass at room temperature. The bonded area was 1 inch by 0.5 inch. The results are reported as the time required for the bond to fail.

Peel adhesion at 180° between the backing and the substrate was measured after wetting out for 20 minutes or 24 hours after the adhesive was applied onto the substrates in accordance to Test Method PSTC (Pressure Sensitive Tape Council, Northbrook, Ill.) 101, section 1.1.1, adapted as follows. All testing was performed at 22° C. and 50% relative humidity. The result reported in Table 2 is the average of three measurements.

Each polymer was isolated and applied onto PET backing layer and tested for shear performance and peel force on either stainless steel or VitroSkin® substrates. The results are shown in Table 2.

TABLE 2
Samples on PET Backing

| | Stainless Steel substrate | | | VitroSkin ® substrate | | |
|---|---|---|---|---|---|---|
| | Shear | Peel 20 min | Peel 24 hr | Shear | Peel 20 min | Peel 24 hr |
| Sample | (min) | (oz-force) | (oz-force) | (min) | (oz-force) | (oz-force) |
| Control | 1327 | 47 | 55 | 8 | 20 | 33 |
| A | 4606 | 52 | 67 | 45 | 17 | 24 |
| B | 394 | 69 | 86 | 19 | 31 | 34 |
| B1 | 10108 | 50 | 54 | — | — | — |
| C | 461 | 72 | 106 | 36 | 45 | 82 |
| C1 | 11754 | 77 | 102 | — | — | — |
| D | 93 | 95 | 126 | 17 | 32 | 65 |
| D1 | 14000 | 40 | 102 | — | — | — |

Shear adhesion values for B1, C1, and D1 samples in Table 2 are significantly higher than Control sample, for both stainless steel and Vitro-Skin® substrates. The addition of HEMA and or MA provides about 4 to about 10-fold improvement over Control. Also, the peel adhesion performances are similar to or improved over the Control sample, for both stainless steel and Vitro-Skin® substrates for Samples A, B, C, and D. The peel adhesion is dependent on the amount of time wetted on the substrate, the type of substrate, and the amount of 2-DMAEMA, HEMA, and/or MA in the polymer.

Similarly, polymers were applied onto Sontara® (Jacob-Holm) backing and shear and peel forces were tested on stainless steel substrates. The results are shown in Table 3.

TABLE 3
Samples on Sontara ® Backing on Stainless steel

| | Stainless Steel substrate | | |
|---|---|---|---|
| | Shear | Peel (oz-force) | Peel (oz-force) |
| Sample | (min) | 20 min | 24 hr |
| Control | 15 | 32 | 47 |
| A | 127 | 32 | 61 |
| B | 118 | 81 | 112 |
| C | 84 | 80 | 104 |
| D | 27 | 76 | 109 |

Shear and peel adhesion performances for Samples A, B, C, and D are also improved for Sontara® backings over Control.

I/We claim:

1. An acrylic polymer prepared from (i) from about 1 to about 10 wt % of a carboxy functional monomer component, (ii) from about 50 to about 90 wt % of a low Tg alkyl acrylate monomer component, (iii) from about 1 to about 20 wt % of a tertiary amine functionalized alkyl acrylate monomer component, and (iv) optionally, up to 50 wt % of a vinyl ester monomer component.

2. The acrylic polymer of claim 1, wherein the (i) a carboxy functional monomer component is selected from the group consisting of acrylic acid, methacrylic acid, itaconic acid, β-carboxyethyl acrylate, and mixtures thereof.

3. The acrylic polymer of claim 1, wherein the (ii) low Tg alkyl acrylate monomer component is selected from the group consisting of methyl acrylate, butyl acrylate, isobutyl acrylate, amyl acrylate, hexyl acrylate, 2-ethylhexyl acrylate, octyl acrylate, isooctyl acrylate, decyl acrylate, dodecyl acrylates, and isomers and mixtures thereof.

4. The acrylic polymer of claim 1, wherein the (iii) a tertiary amine functionalized alkyl acrylate monomer component is 2-dimethyl aminoethyl methacrylate, 2-N-morpholinoethyl acrylate, 2-N-morpholinoethyl methacrylate, 2-diisopropylaminoethyl methacrylate, N-(3-aminopropyl)methacrylamide hydrochloride, N-(3-BOC-aminopropyl)methacrylamide, 2-aminoethyl methacrylate hydrochloride, methacryloyl-L-Lysine, N-[3-(N,N-dimethylamino)propyl] methacrylamide, N-(2-aminoethyl) methacrylamide hydrochloride, N-[2-(N,N-dimethylamino)ethyl]methacrylamide, N-[3-(N,N-dimethylamino)propyl] acrylamide, min. 95%, 2-(N,N-dimethylamino)ethyl acrylate, 2-(N,N-diethylamino)ethyl methacrylate, 2-(tert-butylamino)ethyl methacrylate, 2-(N,N-dimethylamino)ethyl methacrylate, 2-acryloxyethyltrimethylammonium chloride.

5. The acrylic polymer of claim 4, wherein the (iii) a tertiary amine functionalized alkyl acrylate monomer component is 2-dimethyl aminoethyl methacrylate.

6. The acrylic polymer of claim 1, wherein the (iv) vinyl ester monomer component selected from the group consisting of vinyl acetate, vinyl benzoate, vinyl-tert-butylbenzoate, vinyl chloroformate, vinyl cinnamate, vinyl decanoate, vinyl neodecanoate, vinyl pivalate, vinyl propionate, vinyl sterate, vinyl trifloroacetate, vinyl valerate, and mixture thereof.

7. The acrylic polymer of claim 1, further comprising a hydroxy functional monomer component and derivatives thereof.

8. The acrylic polymer of claim 7, wherein the hydroxy functional monomer component is selected from the group consisting of hydroxyethyl acrylate, hydroxypropyl acrylate, hydroxyethyl methacrylate and hydroxypropyl methacrylate, and mixtures thereof.

9. The acrylic polymer of claim 1, further comprising an anhydride functional monomer component and derivatives thereof.

10. The acrylic polymer of claim 9, wherein the anhydride functional monomer component is selected from the group consisting of maleic anhydride, nadic anhydride, methyl maleic anhydride, nadic methyl anhydride or derivatives thereof.

11. The acrylic polymer of claim 1, comprising (i) from about 1 to about 10 wt % of an acrylic acid, (ii) from about 50 to about 90 wt % of a 2-ethylhexyl methacrylate, (iii) from about 1 to about 20 wt % of 2-dimethyl aminoethyl methacrylate and (iv) up to about 50 wt % of vinyl acetate.

12. A pressure sensitive adhesive composition comprising an acrylic polymer prepared from about 1 to about 10 of (i) a carboxy and/or hydroxy functional monomer; (ii) from about 50 to about 90 of a low Tg alkyl acrylate monomer component, (iii) from about 1 to about 20 wt % of a tertiary amine functionalized alkyl acrylate monomer.

13. The pressure sensitive adhesive composition of claim 12 further comprising an additive selected from the group consisting of tackifiers, plasticizers, pigments, fillers, fluorescents, flow agent, wetting agents, surfactants, anti-foaming agents, rheology modifiers, permeation enhancers, stabilizers, antioxidants, and mixtures thereof.

14. The pressure sensitive adhesive composition of claim 12 further comprising a pharmaceutical and/or a nutraceutical active.

15. An article of manufacture comprising the pressure sensitive adhesive of claim 12.

16. The article of claim 15 which is to be adhesively adhered to the skin.

17. The article of claim 15 with a backing substrate and a release liner, wherein the pressure sensitive adhesive is in between the substrate and the release liner.

18. The article of claim 17 which is a tape, a plaster, or a bandage.

19. A method of forming an acrylic polymer comprising the steps of:
a) forming a monomer mixture of 1 to about 10 of (i) a carboxy and/or hydroxy functional monomer; (ii) from about 50 to about 90 of a low Tg alkyl acrylate monomer component, (iii) from about 1 to about 20 wt % of a tertiary amine functionalized alkyl acrylate monomer component in a vessel;
b) adding solvent and a initiator to the vessel;
c) heating the vessel; and
d) isolating the acrylic polymer.
